(19) United States
(12) Patent Application Publication
(10) Pub. No.: US 2022/0274108 A1
(43) Pub. Date: Sep. 1, 2022

(54) MULTI-COLOR SYSTEM FOR REAL TIME PCR DETECTION

(71) Applicant: miDiagnostics NV, Heverlee (BE)

(72) Inventor: Kirill ZINOVIEV, Leuven (BE)

(21) Appl. No.: 17/636,845

(22) PCT Filed: Aug. 21, 2020
(86) PCT No.: PCT/EP2020/073509

§ 371 Date: Feb. 18, 2022

(30) Foreign Application Priority Data
Aug. 23, 2019 (EP) 19193420.7

(51) Int. Cl. B01L 3/00; G02B 21/16; G02B 21/06; G01N 21/64

(52) U.S. Cl. CPC B01L 3/502715; G02B 21/16; G02B 21/06; G01N 21/6428; G01N 21/6456; C12Q 1/686

(57) ABSTRACT

The present inventive concept relates to a system for monitoring a PCR-reaction in a microfluidic reactor. The system comprises: a first light source illuminating the microfluidic reactor through a first excitation light filter providing light of a first excitation wavelength range adapted to excite a first fluorophore in the microfluidic reactor, whereby fluorescent light of a first emission wavelength range is emitted by the first fluorophore; a second light source illuminating the microfluidic reactor through a second excitation light filter providing light of a second excitation wavelength range adapted to excite a second fluorophore in the microfluidic reactor, whereby fluorescent light of a second emission wavelength range is emitted by the second fluorophore; a The system further comprises a first emission filter adapted to transmit fluorescent light of the first emission wavelength range and block fluorescent light of the second emission wavelength range; a second emission filter adapted to transmit fluorescent light of the second emission wavelength range and block fluorescent light of the first emission wavelength range. The system additionally comprises first imaging optics adapted to image the microfluidic reactor onto a first imaging surface, by fluorescent light of the first emission wavelength range whereby the image on the first imaging surface is indicative of a first reaction parameter of the PCR-reaction associated with the first fluorophore; and second imaging optics adapted to image the microfluidic reactor onto a second image surface, by fluorescent light of the second emission wavelength range, thereby monitoring a second reaction parameter of the PCR-reaction associated with the second fluorophore.

### TECHNICAL FIELD

The present inventive concept relates to a system for monitoring a PCR-reaction in a microfluidic reactor. The present inventive concept further relates to a device comprising the system.

### BACKGROUND

Polymerase Chain Reaction (PCR) is commonly used for synthesis or copying of DNA. Evolution of the reaction may be monitored by following a fluorescence signal being proportional to the amount of DNA. DNA fragments differing in length and sequence, may be amplified in the same thermal process, which is known as multiplexing. Each of the fragments may be associated to a different fluorescence wavelength, and single or multiple excitation wavelengths can be used.

It is a problem with multiplex PCR to achieve excitation and detection of fluorophores at different wavelengths.

Other problems with PCR are associated with non-uniformity of the reaction in a reaction vessel and formation of air bubbles in the reaction liquid.

With micro-fluidic systems for PCR having multiple reaction chambers or multiple reaction droplets, there is a need for efficient determination of in which chambers or droplets the reactions are taking place.

There is, thus, a need for miniaturised PCR systems capable of handling and monitoring multiplex reactions, also with a plurality of reaction chambers. Further needs include detection of air bubbles in microfluidic PCR-systems, which may lead to termination of reactions or disruption of fluid transport in the system. Other malfunctions of the PCR-systems, for example related to heating cycles or supply of reagents, is problematic to detect, and typically requires that the PCR is disrupted.

With miniaturised systems where PCR is conducted in micro-droplets, there is a need for efficient counting of droplets. Also, in case of a plurality of parallel reaction compartments, there is need for efficient determination of which compartment comprises active reactions. Solutions may be based on use of standard fluorescence microscopes and multiple colour fluorophores, which systems are bulky and unsuited for miniaturised devices, and which further requires mechanical switching between filtering media to handle multiple colour fluorophores, thus resulting in time consuming and non-continuous detection.

### SUMMARY

According to a first aspect of the present inventive concept there is provided a system for monitoring a PCR-reaction in a microfluidic reactor, the system comprising:

a first light source illuminating the microfluidic reactor through a first excitation light filter providing light of a first excitation wavelength range adapted to excite a first fluorophore in the microfluidic reactor, whereby fluorescent light of a first emission wavelength range is emitted by the first fluorophore,

a second light source illuminating the microfluidic reactor through a second excitation light filter providing light of a second excitation wavelength range adapted to excite a second fluorophore in the microfluidic reactor, whereby fluorescent light of a second emission wavelength range is emitted by the second fluorophore,

a first emission filter adapted to transmit fluorescent light of the first emission wavelength range and block fluorescent light of the second emission wavelength range,

a second emission filter adapted to transmit fluorescent light of the second emission wavelength range and block fluorescent light of the first emission wavelength range,

first imaging optics adapted to image the microfluidic reactor onto a first imaging surface, by fluorescent light of the first emission wavelength range transmitted through the first emission filter, whereby the image on the first imaging surface is indicative of a first reaction parameter of the PCR-reaction associated with the first fluorophore, and

second imaging optics adapted to image the microfluidic reactor onto a second image surface, by fluorescent light of the second emission wavelength range transmitted through the second emission filter, thereby monitoring a second reaction parameter of the PCR-reaction associated with the second fluorophore.

According to a second aspect of the present inventive concept there is provided a device comprising the system according to the first aspect.

### BRIEF DESCRIPTION OF THE DRAWINGS

The above, as well as additional objects, features and advantages of the present inventive concept, will be better understood through the following illustrative and non-limiting detailed description, with reference to the appended drawings. In the drawings like reference numerals will be used for like elements unless stated otherwise.

FIG. 1 is a schematic illustration of a system for monitoring a PCR-reaction in a microfluidic reactor.

FIG. 2 is a schematic illustration of an embodiment of a system for monitoring PCR-reactions in a microfluidic reactor.

FIG. 3 is a schematic illustration of an embodiment of a system for monitoring PCR-reactions in a microfluidic reactor.

FIG. 4 is a schematic illustration of an embodiment of a system for monitoring PCR-reactions in a microfluidic reactor.

FIG. 5 is a schematic illustration of embodiments of a system for monitoring PCR-reactions in a microfluidic reactor illustrating different positioning of light sources, filters, and optics.

### DETAILED DESCRIPTION

In view of the above, it would be desirable to achieving systems for monitoring a PCR-reaction in a microfluidic reactor, which are not compromised by problems associated with prior art. An objective of the present inventive concept is to address this issue and to provide solutions to at least one problem or need related to prior art. Further and alternative objectives may be understood from the following.

Disclosures herein relating to one inventive aspect of the inventive concept generally may further relate to one or more of the other aspect(s) of the inventive concept.

According to a first aspect of the present inventive concept there is provided a system for monitoring a PCR-reaction in a microfluidic reactor, the system comprising:

a first light source illuminating the microfluidic reactor through a first excitation light filter providing light of a first excitation wavelength range adapted to excite a first fluorophore in the microfluidic reactor, whereby fluorescent light of a first emission wavelength range is emitted by the first fluorophore,

a second light source illuminating the microfluidic reactor through a second excitation light filter providing light of a second excitation wavelength range adapted to excite a second fluorophore in the microfluidic reactor, whereby fluorescent light of a second emission wavelength range is emitted by the second fluorophore,

a first emission filter adapted to transmit fluorescent light of the first emission wavelength range and block fluorescent light of the second emission wavelength range,

a second emission filter adapted to transmit fluorescent light of the second emission wavelength range and block fluorescent light of the first emission wavelength range,

first imaging optics adapted to image the microfluidic reactor onto a first imaging surface, by fluorescent light of the first emission wavelength range transmitted through the first emission filter, whereby the image on the first imaging surface is indicative of a first reaction parameter of the PCR-reaction associated with the first fluorophore, and

second imaging optics adapted to image the microfluidic reactor onto a second image surface, by fluorescent light of the second emission wavelength range transmitted through the second emission filter, thereby monitoring a second reaction parameter of the PCR-reaction associated with the second fluorophore.

The system comprising a first light source and a second light source associated with a first and a second excitation light filter respectively allows for continuous and simultaneous illumination of the microfluidic reactor at two different wavelengths, and, thereby, continuous and simultaneous excitation of two different type of fluorophores.

The system further comprising a first emission filter and a second emission filter, allows for continuous and simultaneous transmittal of excited light from the two types of fluorophores.

The combination of the first light source and the second light source associated with the first and the second excitation light filter respectively, with the first emission filter and the second emission filter, respectively, enables efficient and continuous monitoring of two types of fluorophores simultaneously, and, thereby, continuous and independent monitoring of, for example, two reaction parameters or two reactions. The provision of a plurality of light sources instead of one, allows for a plurality of fluorophores to be used with the system without a need for switching between different excitation light filters. Thus, continuous, and parallel monitoring of more than one fluorophore or reaction parameter is allowed.

Each imaging optics being associated with one of the emission wavelengths, allows imaging of each type of fluorophore spatially separated on the imaging surface.

The imaging surface enables spatial information from the PCR-reaction to be monitored. For example, it may be monitored at which locations of a microfluidic system reactions occur. Spatial information together with quantitative analysis obtainable with fluorescent detection allows quantitative analysis at spatially different locations of the microfluidic reactor.

The system, thus, allows simultaneous and continuous analysis of a plurality of reaction parameters each associated with one type of fluorophore, with spatial information relating to locations of the microfluidic system. Thereby, it is made possible to, for example, identify where in the system a specific PCR-reaction occurs, even for multiplex PCR. Further, variations in a PCR-reaction may be associated with, for example, variations of reaction parameters identifiable with fluorophores, such as temperatures, concentration of reactants or pH.

The first imaging surface and the second imaging surface may each correspond to a first portion and a second portion, respectively, of a single image sensor; or a first image sensor, and a second image sensor, respectively, wherein the first and the second portions of the image sensor; or the first image sensor and the second image sensor; each are adapted to provide a digital representation of the image of the corresponding imaging surface. Thereby, each type of fluorophore may efficiently be monitored. Further, separate imaging may be obtained for each fluorophore.

The single image sensor or the first and second image sensors may be any suitable image sensor, such as image sensors known in the art for sensing of images. For example, the image sensor may be of a type selected from the group consisting of CMOS imaging sensors, sCMOS imaging sensors and CCD sensors.

The single image sensor may be associated with two, or more, imaging pixels; and the first and second image sensors may each be associated with one or more imaging pixels. Thereby, resolution between the first and the second excitation wavelengths may be realised.

The microfluidic reactor may further comprise microfluidic channels for transport of, for example, reactants, reaction products, buffers, fluids, additives, and cleaning fluids. Actuating of liquids to, from, and within the system may suitably be arranged by active or passive pumps, which pumps further may be integrated in the system or connectable to the system.

The first and the second light sources may be arranged to provide light continuously, thereby allowing continuous monitoring of the first reaction parameter and the second reaction parameter.

The first and second light sources, and any optional and additional light sources, may be of LED type or of a broad-spectrum type.

It shall be appreciated that, with the described system comprising the filters and the first and second imaging optics, and the first and second imaging surfaces, it is possible to continuously and in parallel illuminating the microfluidic reactor with a first and a second emission wave lengths. Thereby there is no need for switching between excitation light filters. Further, a continuous monitoring of PCR reactions and spatial imaging of the microfluidic reactor may be realised. Embodiments of the present invention may thereby benefit from continuous monitoring of PCR-reactions.

With providing light continuously is intended to describe that the light sources are not switched on and off repeatedly. The light sources may be switched on and off at a beginning and at an end of the monitoring, and the light sources may be switched off during periods of an analysis or PCR-reaction, and still be considered to be continuous as used herein. With present embodiments it is realisable to have the first and the second light sources switched on simultaneously or in parallel.

The first and second light sources, the first and second emission filters, and the first and second imaging optics may be arranged opposing the same side of the microfluidic reactor. Thereby, the system may be provided in a compact fashion, and provide efficient imaging of fluorescent light with reduced disturbance from excitation light or stray light.

The first and the second fluorophores may be selected such that the first emission wavelength range and the second emission wavelength range are not overlapping. Thereby detection interference may be avoided or reduced.

The microfluidic reactor may comprise a translucent wall portion arranged to allow imaging of at least a portion of the microfluidic reactor. Thereby, for example, spatial information on the PCR-reactions are efficiently facilitated.

The translucent wall portion may be translucent to a wavelength interval comprising the first and the second excitation wavelengths and the first and the second emission wavelengths.

The first emission filter may further be adapted to block light outside of the first emission wavelength range, and the second emission filter may further be adapted to block light outside the second emission wavelength range.

The first fluorophore may be associated with DNA produced in the PCR-reaction, whereby the image on the first imaging surface is indicative of an amount of produced DNA. For example, the first fluorophore may be a fluorescent label bound to the DNA.

During PCR of several different DNA sequences, such as during multiplex PCR, the first fluorophore may be associated with a first DNA sequence. A second or a third fluorophore may be associated with a second DNA sequence or another reaction parameter. Thereby, it is enabled to monitor production of different DNA sequences during PCR.

The first and the second reaction parameters may be different and each may be selected from the group consisting of: a temperature in the microfluidic reactor, an amount of produced DNA, an amount of a reactant, and pH. It is to be understood that the skilled person may apply the system to other parameters as well. At least one of the reaction parameters may be an amount of produced DNA.

The reaction parameter being temperature may be realised by, for example, a temperature sensitive or dependent fluorophore.

The reaction parameter being pH may be realised by, for example, a pH-sensitive or pH-dependent fluorophore.

The system may further comprise first excitation optics and second excitation optics, wherein the first excitation optics are arranged to transfer light from the first light source to the first excitation light filter, and the second excitation optics are arranged to transfer light from the second light source to the second excitation light filter.

The excitation optics and the imaging optics each may comprise an arrangement comprising one or more lenses.

The system may further comprise a third light source illuminating the microfluidic reactor through a third excitation light filter providing light of a third excitation wavelength range adapted to excite a third fluorophore in the microfluidic reactor, whereby fluorescent light of a third emission wavelength range is emitted by the third fluorophore,

a third emission filter adapted to transmit fluorescent light of the third emission wavelength range and block fluorescent light of the first and the second emission wavelength ranges, and

third imaging optics adapted to image the microfluidic reactor onto a third imaging surface, by fluorescent light of the third emission wavelength range transmitted through the third emission filter, whereby the image on the third imaging surface is indicative of a third reaction parameter of the PCR-reaction associated with the third fluorophore,

wherein the first and the second emission filters further are adapted to block fluorescent light of the third emission wavelength range.

The system may further comprise a fourth to tenth, or more, light sources, emission filters, and imaging optics, thereby allowing additional monitoring of a fourth to a tenth, or more, reaction parameters.

In embodiments having a system comprising more than a first and a second light sources, such as an additional third or additionally a fourth to a tenth or more light sources, the system further comprises optics, filters and fluorophores individually associated with each light sources in analogy with the first and second light sources and the description above relating the third light source.

The first and second, or more, fluorophores of the system may be different in that they each are associated with excitation wavelengths and emission wavelengths differing from the others. More than one different fluorophore, such as a first and a second, may be part of, such as bound to, a single structure, such as a molecule or particle.

The microfluidic reactor may comprise a first and a second reaction compartment.

The microfluidic reactor may comprise a first and a second reaction compartment, wherein the first imaging optics further is adapted to image the first reaction compartment on the first imaging surface, and the second imaging optics further is adapted to image the second reaction compartment on the second imaging surface. Thereby, parallel reactions in separate compartments may be monitored. An array of reaction compartments on a microfluidic device may be monitored simultaneously.

The reaction compartment may, for example, be a cell, a well, a chamber or a channel.

The system may further comprise a processing device. The processing device may be used for temperature controlling the microfluidic reactor, controlling the light sources, and/or controlling image capturing. The processing device may also be used to process data and/or transfer data to a monitoring device.

According to a second aspect of the present inventive concept there is provided a device comprising the system according to the first aspect.

The second aspect may generally have the same features and advantages as the first aspect. To avoid undue repetition, reference is thereby made to the sections above which are equally applicable to the device. It is further noted that the inventive concepts relate to all possible combinations of features unless explicitly stated otherwise.

Exemplary embodiments will now be described more fully hereinafter with reference to the accompanying drawings. The inventive concepts may, however, be embodied in many different forms and should not be construed as limited to the embodiments set forth herein; rather, these embodiments are provided for thoroughness and completeness, and fully convey the scope of the inventive concepts to the skilled person.

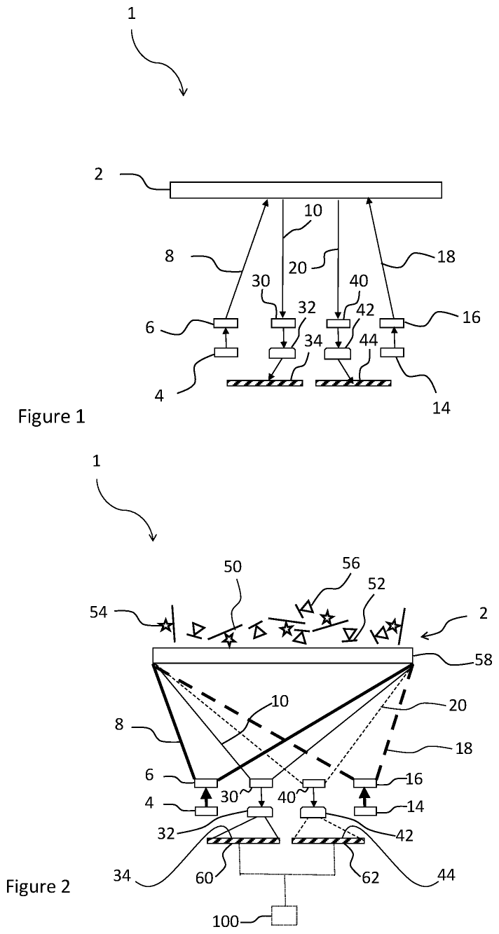

FIG. 1 schematically illustrates a system 1 for monitoring a PCR-reaction in a microfluidic reactor 2. The system 1 comprises a first light source 4 illuminating the microfluidic reactor 2 through a first excitation light filter 6 providing light of a first excitation wavelength range 8 adapted to excite a first fluorophore (not illustrated) in the microfluidic reactor 2, whereby fluorescent light of a first emission wavelength range 10 is emitted by the first fluorophore. A second light source 14 illuminating the microfluidic reactor 2 through a second excitation light filter 16 providing light of a second excitation wavelength range 18 adapted to excite a second fluorophore in the microfluidic reactor 2, whereby fluorescent light of a second emission wavelength range 20 is emitted by the second fluorophore. A first emission filter 30 is adapted to transmit fluorescent light of the first emission wavelength range 10 and block fluorescent light of the second emission wavelength range 20. A second emission filter 40 is adapted to transmit fluorescent light of the second emission wavelength range 20 and block fluorescent light of the first emission wavelength range 10. First imaging optics 32 is adapted to image the microfluidic reactor 2 onto a first imaging surface 34, by fluorescent light of the first emission wavelength range 10 transmitted through the first emission filter 30, whereby the image on the first imaging surface 34 is indicative of a first reaction parameter of the PCR-reaction associated with the first fluorophore. A second imaging optics 42 adapted to image the microfluidic reactor 2 onto a second image surface 44, by fluorescent light of the second emission wavelength range 20 transmitted through the second emission filter 40, thereby monitoring a second reaction parameter of the PCR-reaction associated with the second fluorophore.

Excitation light and emission light have been schematically illustrated in FIG. 1 by arrows in an attempt to improve understanding of the system 1, although the arrows may not correspond to or illustrate a realistic beam-width or behaviour of the light.

Although the first and the second image surfaces 34, 44 may be viewed, as illustrated in FIG. 1, as separated surfaces, they may be portions of a single image sensor, or, correspond to separate sensors.

The spectra of the excitation wavelength ranges may not overlap with the spectra of the emission wavelength range. Thereby, imaging disturbance caused by stray light or light not associated with emission may be reduced or avoided.

The system may further comprise a heating arrangement (not illustrated), configured to heat the microfluidic reactor or one or more portions of the microfluidic reactor. Thereby heating cycles for the PCT-reaction may be realised.

Although not illustrated in FIG. 1, the PCR-reactions may be conducted in a plurality, such as an array, of microfluidic compartments. With a system of the present inventive concept, it may efficiently be determined which reaction compartments of the plurality of compartments comprises progressing or active PCR-reaction. The microfluidic compartment may be a micro-droplet, and the system may comprise an array of micro-droplets.

FIG. 2 schematically illustrates use of the system 1 for monitoring of multiplex PCR. In the illustrated example, two types of DNA molecules, a first DNA 50 illustrated by solid lines and a second DNA 52 illustrated by dotted lines, differing for example in length and or/sequence are copied. In the example, PCR is performed on the first DNA 50 and the second DNA 52 simultaneously in a microfluidic reaction compartment of the microfluidic reactor 2. The reaction compartment of the example is a compartment on a microfluidic chip. Fluids, monomers, and any other suitable additives for the reactions are not illustrated in an attempt to improve clarity. The first DNA 50 is associated with a first fluorophore 54 and the second DNA 52 with a second fluorophore 56. Further illustrated is a first and a second light source 4, 14, illuminating the microfluidic reactor 2 through first and second excitation light filters 6, 16, respectively. In the example, light of a first and a second excitation wavelength range 8, 18 provided by the light sources are illuminating the entire reaction chamber through a translucent bottom portion 58, thereby, fluorophores 54, 56 throughout the microfluidic reactor 2 are illuminated by light. The first and second fluorophores 54, 56 and the first and second excitation light filters 4, 14 are selected such that the fluorophores are excited by the light, resulting in fluorescent light of a first and a second emission wavelength range 10, 20, being emitted by the first and second fluorophores 54, 56, respectively. The emitted light of the fluorophores 54, 56 is in the example associated with the concentration of produced first and second DNAs 50, 52, which concentrations, thus, may be determined. For determination of the concentrations, for example a standard curve may be used. At least a part of the emitted light will shine through the bottom portion 58, and thereby, fluorescent light of the first and second emission wavelength ranges 10, 20 will reach the first and second emission filters 30, 40, which, based on known data of the fluorophores are adapted to transmit fluorescent light of the first and second emission wavelength ranges 10, 20, respectively. The light sources, fluorophores, and filters are further selected such that excitation light do not overlap with emission light. At least portions of the emitted lights thereby reach the first and second emission filters 30, 40, which are adapted to transmit fluorescent light of the first and second emission wavelength ranges, respectively, and block fluorescent light of the second and first emission wavelength ranges, respectively. Thus transmitted light thereafter reaches first and second imaging optics 32, 42, which image the microfluidic reactor onto first and second imaging surfaces 34, 44. Thereby, fluorescent light from the first fluorophores 54 from the entire microfluidic reactor 2 will be imaged on the first imaging surface 34 of a first image sensor 60, and fluorescent light from the second fluorophores 56 from the entire microfluidic reactor 2 will be imaged on the second imaging surface 44 of a second image sensor 62, wherein each sensor is adapted to provide a digital representation of the image of the corresponding imaging surface. The first and second image sensors are each associated with one or more imaging pixels, for example up to a hundred, a thousand or millions of pixels. Thereby, an image of the reaction chamber where fluorophores, and thus indirectly DNA is visualised may be provided with a resolution sufficient for providing, by way of example, spatial information. It may, for example, be visualised or determined on which portions of a chip PCR-reactions are active, or non-active. This in combination with the possibility to determine reaction parameters such as temperature and/or pH enables associations between activity, or progress, of PCR-reaction and temperature or pH. Further, for a microfluidic reactor 2 comprising a plurality or reaction locations, such as reaction compartments, channels, or micro-droplets, the spatial information allowed with the plurality of pixels may provide information on activity in the individual reaction locations. Further illustrated in FIG. 2 is a processing device 100, which may be connectable to or included in a system 1 according to the inventive concepts, for example for temperature controlling the microfluidic reactor 2, controlling the light sources, and/or controlling image capturing.

In the example illustrated with reference to FIG. 2, a single reaction compartment comprised on the microfluidic reactor 2 was illustrated to comprise the PCR-reaction that was being monitored. Two light sources were used in monitoring of the PCR-reactions. The system may alternatively use two light sources for monitoring of two portions of the microfluidic reactor. For example, the microfluidic reactor 2 may comprise a first and a second reaction compartment for copying of the first DNA 50 and the second DNA 52, respectively.

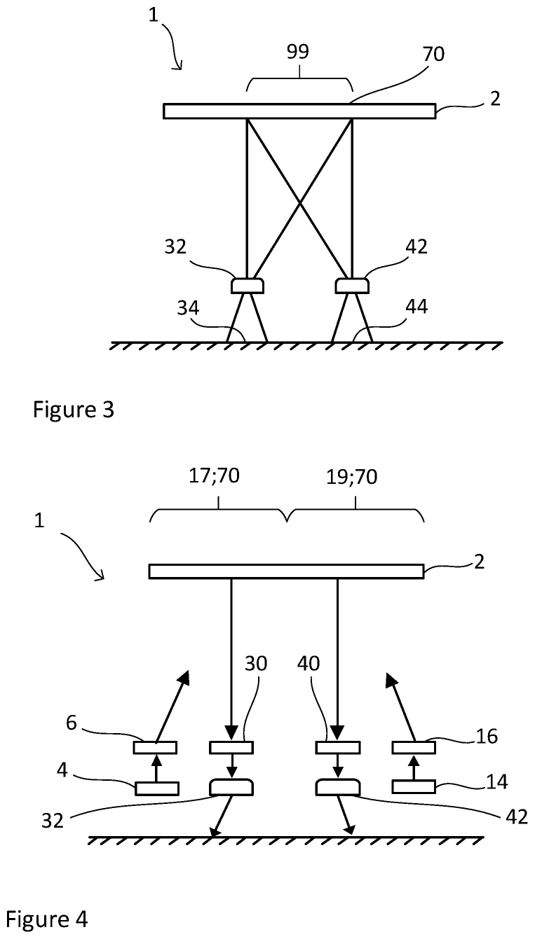

According to one example of an embodiment of the present inventive concept as illustrated in FIG. 3, the system 1 may have first imaging optics 32 and second imaging optics 42, both adapted to image a same area 99, or portion, of the microfluidic reactor 2, for example a reaction compartment 70, on the first imaging surface 34 and the second imaging surface 44, respectively. The first and second emission filters 30, 40, and the first and second excitation light filters 6, 16 are not illustrated. The first and a second light source 4, 14 are illuminating the same area, or portion, of the microfluidic reactor 2. The first fluorophore 54 and the second fluorophore 56 (not illustrated) may be selected to determine, for example, concentration of produced DNA and concentration or presence of monomers for the PCR-reaction, respectively. With such a system progress of PCR-reactions may be determined and related to the concentration of monomers. For example, if the DNA concentration is not increasing or no presence of DNA is indicated, and there is low or no presence of monomer indicated, it may be determined that there may be problems with provision of monomer. Alternatively, the second fluorophore may be selected to indicate a temperature, or there may be a third light source and fluorophore present which may be used for monitoring of temperature in addition to or parallel to the monitoring of DNA and monomer concentration. It may then be determined if an unexpected or undesired concentration of DNA is linked to temperature and/or concentration of monomer.

FIG. 4 schematically illustrates a system 1 with first and second portions 17, 19 of the microfluidic reactor 2 being monitored individually. Progress of PCR reaction in each portion may thereby be monitored. The first and second portions may be first and second reaction compartments 70. Further illustrated are first and second light sources 4, 14, each illuminating at least a part of the first and second portions 17, 19, respectively. They may illuminate the entire microfluidic reactor. Yet further illustrated are first and second excitation light filters 6, 16; first and second emission filters 30, 40; first imaging optics 32 adapted to image the first portion 17, for example the first reaction compartment, on the first imaging surface 3; and second imaging optics 42 adapted to image the second portion 19, for example the second reaction compartment, on the second imaging surface 44. With such a system 1, for example a microfluidic reactor 2 comprising a first and a second reaction compartments 70 for PCR reaction of a first and a second DNA, respectively may be monitored. Different portions within a microfluidic compartment 70 may alternatively be monitored. The progress of the PCR reaction may optionally be related to a determined third reaction parameter, such as temperature, pH, or concentration of a reagent. It may be determined, for example, that the PCR reaction in one or more of the reaction compartments 70 is malfunctioning, for example by determining that no DNA has been produced or that the production of DNA is not following a predetermined pattern or that the concentration of produced DNA is unexpected. Additional information concerning, for example, the temperature being out of desired range may provide an indication of reasons for the malfunctioning and further indicate that the temperature should be adjusted.

According to another embodiment of the present inventive concept, a microfluidic reactor 2 may have a plurality of microfluidic reaction compartments 70, for example the microfluidic reactor 2 may comprise 1 to 100, or more, microfluidic reaction compartments 70. Embodiments of the present inventive concept allows PCR reactions of all or some of the microfluidic compartments to be monitored. It may, for example, be determined in which of the compartments PCR occurs at any given time or over a period of time. Further, bubble formation may be identified. For example, qualitative and/or quantitative measurements of produced DNA may be determined and the development of the PCR in each or a group of compartments may be determined, such as by monitoring fluorophores associated with production of DNA. Unexpected development may be linked or related to a reactions parameter, for example an unexpected low production of DNA in one or more compartments or group of compartments may be linked to undesired temperatures. The system may beneficially be used also for microfluidic reactors 2 comprising a plurality, such as one or more arrays, of micro-droplets functioning as reactors.

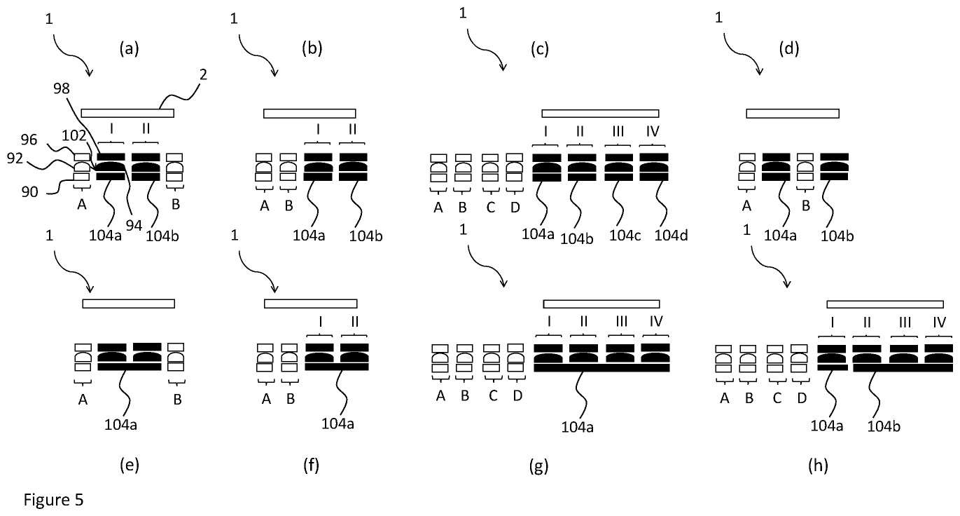

FIGS. 5a-h illustrate embodiments of the system 1 according to the present inventive concept. FIGS. 5a-h illustrates the microfluidic reactor 2 and different examples of positioning of light sources 90, optional excitation optics 92, imaging optics 94, excitation light filters 96, emission filters 98, imaging surfaces 102 and imaging sensors 104. A first to fourth group of a light source 90, an excitation optics 92, and an excitation light filter 96 are indicated by A to D, respectively. A first to fourth group of emission filter 98, imaging optics 94, and imaging surface 102 are indicated by I-IV, respectively. First to fourth image sensors 104 are indicated by 104a-d.

In the examples illustrated in FIGS. 5a-h, each imaging surface corresponds to a single image sensor, while in the examples illustrated by FIGS. 5e-g, each imaging surface corresponds to a portion of a single image sensor. FIG. 5h illustrates an example combining one imaging surface corresponding to a single image sensor, with a plurality of imaging surfaces corresponding to a plurality of portions of a single imaging sensor.

1. A system for monitoring a PCR-reaction in a microfluidic reactor, the system comprising:
a first light source illuminating the microfluidic reactor through a first excitation light filter providing light of a first excitation wavelength range adapted to excite a first fluorophore in the microfluidic reactor , whereby fluorescent light of a first emission wavelength range is emitted by the first fluorophore,
a second light source illuminating the microfluidic reactor through a second excitation light filter providing light of a second excitation wavelength range adapted to excite a second fluorophore in the microfluidic reactor, whereby fluorescent light of a second emission wavelength range is emitted by the second fluorophore,
a first emission filter adapted to transmit fluorescent light of the first emission wavelength range and block fluorescent light of the second emission wavelength range,
a second emission filter adapted to transmit fluorescent light of the second emission wavelength range and block fluorescent light of the first emission wavelength range,
first imaging optics adapted to image the microfluidic reactor onto a first imaging surface, by fluorescent light of the first emission wavelength range transmitted through the first emission filter, whereby the image on the first imaging surface is indicative of a first reaction parameter of the PCR-reaction associated with the first fluorophore, and
second imaging optics adapted to image the microfluidic reactor onto a second image surface, by fluorescent light of the second emission wavelength range transmitted through the second emission filter, thereby monitoring a second reaction parameter of the PCR-reaction associated with the second fluorophore.

2. The system according to claim 1, wherein the first imaging surface and the second imaging surface each corresponds to
a first portion and a second portion, respectively, of a single image sensor; or
a first image sensor, and a second image sensor, respectively,
wherein the first and the second portions of the image sensor; or the first image sensor and the second image sensor; each are adapted to provide a digital representation of the image of the corresponding imaging surface.

3. The system according to claim 2, wherein the single image sensor is associated with two, or more, imaging pixels; and the first and second image sensors each are associated with one or more imaging pixels.

4. The system according to claim 1, wherein the first and the second light sources are arranged to provide light continuously, thereby allowing continuous monitoring of the first reaction parameter and the second reaction parameter.

5. The system according to claim 1, wherein the first and second light sources, the first and second emission filters, and the first and second imaging optics are arranged opposing the same side of the microfluidic reactor.

6. The system according to claim 1, wherein the first and the second fluorophores are selected such that the first emission wavelength range and the second emission wavelength range are not overlapping.

7. The system according to claim 1, wherein the microfluidic reactor comprises a translucent wall portion arranged to allow imaging of at least a portion of the microfluidic reactor.

8. The system according to claim 1, wherein
the first emission filter further is adapted to block light outside of the first emission wavelength range, and
the second emission filter further is adapted to block light outside the second emission wavelength range.

9. The system according to claim 1, wherein the first fluorophore is associated with DNA produced in the PCR-reaction, whereby the image on the first imaging surface is indicative of an amount of produced DNA.

10. The system according to claim 1, wherein the first and the second reaction parameters are different and each is selected from the group consisting of: a temperature in the microfluidic reactor, an amount of produced DNA, an amount of a reactant, and pH.

11. The system according to claim 1, wherein the system further comprising first excitation optics and second excitation optics, wherein
the first excitation optics are arranged to transfer light from the first light source to the first excitation light filter, and
the second excitation optics are arranged to transfer light from the second light source to the second excitation light filter.

12. The system according to claim 1, wherein the system further comprising
a third light source illuminating the microfluidic reactor through a third excitation light filter providing light of a third excitation wavelength range adapted to excite a third fluorophore in the microfluidic reactor, whereby fluorescent light of a third emission wavelength range is emitted by the third fluorophore,
a third emission filter adapted to transmit fluorescent light of the third emission wavelength range and block fluorescent light of the first and the second emission wavelength ranges, and
third imaging optics adapted to image the microfluidic reactor onto a third imaging surface, by fluorescent light of the third emission wavelength range transmitted through the third emission filter, whereby the image on the third imaging surface is indicative of a third reaction parameter of the PCR-reaction associated with the third fluorophore,
wherein the first and the second emission filters further are adapted to block fluorescent light of the third emission wavelength range.

13. The system according to claim 1, wherein the microfluidic reactor comprises a first and a second reaction compartment,
wherein
the first imaging optics further is adapted to image the first reaction compartment on the first imaging surface, and
the second imaging optics further is adapted to image the second reaction compartment on the second imaging surface.

14. The system according to claim 1, the system further comprising a processor for controlling the monitoring.

15. A device comprising the system according to claim 1.
